(19) United States
(12) Patent Application Publication
(10) Pub. No.: US 2011/0020813 A1
(43) Pub. Date: Jan. 27, 2011

(54) ADVANCED PATHOGEN DETECTION AND SCREENING

(75) Inventors: CRYSTAL ICENHOUR, CHARLOTTESVILLE, VA (US); ELIZABETH J. COLEMAN, CHARLOTTESVILLE, VA (US); BRIAN V. LOYAL, CHARLOTTESVILLE, VA (US)

Correspondence Address: MCGUIREWOODS, LLP, 1750 TYSONS BLVD, SUITE 1800, MCLEAN, VA, 22102, US

(73) Assignee: PHTHISIS DIAGNOSTICS LLC, CHARLOTTESVILLE, VA (US)

(21) Appl. No.: 12/791,811

(22) Filed: Jun. 1, 2010

Related U.S. Application Data
(60) Provisional application No. 61/182,362, filed on May 29, 2009.

(51) Int. Cl. C12Q 1/68

(52) U.S. Cl. 435/6

(57) ABSTRACT

Disclosed is a rapid, dual purpose, PCR-based method for identifying two or more pathogens, including Giardia and/or Cryptosporidium, in an extracted sample, such as stool or environmental (soil, water) isolates, in individual real-time PCR reactions. This method is of particular utility for clinical, veterinary, and environmental testing applications. The present methods are more sensitive than conventional ELISA or IFA-based methods of detection. An internal control (IC) for use in the PCR based nucleic acid detection method is also disclosed.

### CROSS-REFERENCE TO RELATED APPLICATIONS

The application claims priority under 35 U.S.C. §119(c) to provisional U.S. Patent Application No. 61/182,362, filed on May 29, 2009, the disclosure of which is expressly incorporated by reference herein in its entirety.

### GOVERNMENT INTEREST

The United States Government may own rights in the present disclosure pursuant to NIH I R41 AI069598-01 and/or 2 R42 AI069598-02.

### BACKGROUND

1. Field of the Invention

The present disclosure relates to a PCR method, which enables in individual assays the detection of any combination of pathogens, particularly Giardia and Cryptosporidium.

2. Description of the Related Art

Giardia is a protozoan parasite that is a major cause of diarrheal disease worldwide. The most common species of Giardia is G. lamblia, which is the most common pathogenic parasite in North America (Meyer and Jarrol (1980) Am. J. Epidemiol. 3: 1-12). Giardia has two life stages. The trophozoite stage inhabits the small intestine of host animals, moving about using flagella. A suction disk allows the trophozoite to attach to the wall of the intestine while it feeds on mucous secretions. The second life stage, the cyst, has a stronger outer layer, and thus better able than the trophozoite to survive outside of the host while passing from host to host. Transmission is typically through Giardia-contaminated water supplies (Meyer and Jarrol, supra.), or person to person (Black et al. (1977) Pediatrics 60: 486-491).

The cytoskeleton of G. lamblia trophozoites contain a group of 29-38 kDa proteins known as giardins (Peattie et al. (1989) J. Cell Biol. 109: 2323-2335). Nucleic acid sequences are known for several of the giardins, including alpha-1-giardin and alpha-2-giardin, which are 81% identical at the nucleic acid level and have amino acid sequences that are 77% identical (Alonso and Peattie (1992) Mol. Biochem. Parasitol. 50: 95-104). The alpha-1-giardin has been identified on the membrane and disk of G. lamblia trophozoites (Wenman et al. (1993) Parasitol. Res. 79: 587-592).

Traditionally, Giardia infection is diagnosed by microscopic detection of ova and parasites (O&P) in stools, which is a laborious process. More recently developed methods for Giardia diagnosis include serologic tests for anti-Giardia antibodies. Little correlation was found, however, between the presence of anti-Giardia antibodies in the serum and active Giardia infection. Other diagnostic methods involve detection of Giardia antigens in stool samples. For example, Green et al. discuss the use of an affinity-purified antiserum raised by inoculating rabbits with whole trophozoites or disrupted trophozoites and cysts (Green et al. (1985) Lancet 2: 691-693). Other groups have described the use of monospecific antibodies that bind to a 65 kDa antigen that is shed in the stool of Giardiasis patients (Rosoff and Stibbs (1986) J. Clin. Microbiol. 24: 1079-1083; U.S. Pat. No. 5,503,983; Stibbs (1989) J. Clin. Microbiol. 27: 2582-2588; Rosoff et al. (1989) J. Clin. Microbiol. 27: 1997-2002). Monoclonal antibodies that bind to two species of Giardia cyst wall constituents are discussed in Lujan et al. (1995) J. Biol. Chem. 270: 29307-29313. ELISA assays for G. lamblia are discussed in, for example, Nash et al. (1987) J. Clin. Microbiol. 25: 1169-1171; Stibbs et al. (1988) J. Clin. Microbiol. 26: 1665-1669; and Ungar et al. (1984) J. Infect. Dis. 149: 90-97.

Previously described assays for detecting Giardia infection often have shortcomings. For example, the assay of Ungar et al. was reported to fail to detect 8% of positive samples and cannot be read by direct visual inspection (Green et al., supra.).

Giardia lamblia is the only species of the genus that is known to cause disease in humans. Some controversy still surrounds the systematics of the species which is also referred to as Giardia duodenalis or Giardia intestinalis (Lu et al. 1998 Molecular comparison of Giardia lamblia isolates. Int. J. Parasitol. 28: 1341-1345). Other representatives of the genus Giardia described to date are Giardia agilis from amphibians and Giardia muris from rodents, birds and reptiles (Meyer 1994 Giardia as an organism. P 3-13. In: RCA. Thompson, J. A. Reynoldsen, A. J. Lymbery (eds.) Giardia: From molecules to disease. CAB International, Wallingford, Oxon, UK), Giardia ardea from herons (Erlandsen et al. 1990 Axenic culture and characterization of Giardia ardea from the great blue heron (Ardea herodias) (J. Parasitol. 76: 717-724) and Giardia microti from muskrats and voles (van Keulen et al. 1998). The sequence of Giardia small subunit rRNA shows that voles and muskrats are parasitized by a unique species Giardia microti. J. Parasitol. 84: 294-300). Monoclonal antibodies (mabs) are the most important and widely applied tool for detection of Giardia cysts in water samples. The vast majority of commercially available antibodies show a lack of specificity as the antibodies detect all Giardia species including species that do not infect humans. As a positive antibody reaction does not allow any conclusion regarding the viability (infectivity) of the cysts, viability stains (DAPI, PI) have to be used in conjunction with antibodies.

Cryptosporidium is detected by light microscopic examination of focal smears for oocysts or by polymerase chain reaction (PCR) analysis of fecal samples using Cryptosporidium specific oligonucleotide primers. For example, U.S. Pat. No. 5,770,368 to De Leon et al. discloses a method for detecting encysted forms of Cryptosporidium that are viable and infectious. The method involves isolating oocysts, inducing transcription of the heat shock protein (HSP) genes, and detecting the induced transcripts by RT-PCR. Alternatively, infectivity is determined by cultivating the Cryptosporidium on susceptible cells and either amplifying HSP DNA from infected cells by PCR or induced HSP transcription and detecting the induced transcripts by RT-PCR.

PCR is generally considered the most sensitive and rapid method for detecting nucleic acids of a pathogen in a particular sample. PCR is well known in the art and has been described in U.S. Pat. No. 4,683,195 to Mullis et al., U.S. Pat. No. 4,683,202 to Mullis, U.S. Pat. No. 5,298,392 to Atlas et al., and U.S. Pat. No. 5,437,990 to Burg et al. In the PCR step, oligonucleotide primer pairs for each of the target pathogens are provided wherein each primer pair includes a first nucleotide sequence complementary to a sequence flanking the 5′ end of the target nucleic acid sequence and a second nucleotide sequence complementary to a nucleotide sequence flanking the 3′ end of the target nucleic acid sequence. The nucleotide sequences of each oligonucleotide primer pair are specific to particular pathogen to be detected and do not cross-react with other pathogens.

There are multiple non-interchangeable real time PCR platforms in use in clinical laboratories. Some, such as the Roche COBAS and HIV RNA amplification machines, are closed platform, sample-in-result-out devices. By design, these are inflexible and not amenable to adapting to other purposes (such as a de novo Giardia or Cryptosporidium assay).

The LightCycler 2.0 is the major open platform machine in use in clinical laboratories. It is logical to develop the assay for compatibility with a carefully chosen few instruments to increase usability. Therefore, in addition to the LightCycler, an assay should also be adaptable to the Cepheid SmartCycler, Applied Biosystems AB17300/7500, and other instruments as adopted by clinical laboratories. Other candidates are the Corbett Roto-gene and the BioRad iCycler.

A PCR assay used by a clinical laboratory needs to have an internal control DNA template that amplifies to confirm that overwhelming PCR inhibition did not occur. This is particularly critical for a stool-based assay due to the complexity of this specimen.

Currently-used clinical diagnostic and water quality tests for Giardia and Cryptosporidium are time-consuming, difficult to perform, and not as sensitive or specific as desired. Clinical diagnostic labs use ELISA and/or IFA microscopic identification to diagnose Cryptosporidium and Giardia. Unfortunately, ELISA is not as sensitive or specific as DNA-based diagnostics. Additionally, ELISA-based tests can take more than 4 hours to perform. IFA microscopy is costly, involves significant technician time, and provides an unsatisfactory limit of detection (low sensitivity).

In water quality testing, most labs use high volume filtration combined with IFA microscopy. The costs of these tests are quite high, and also require highly skilled personnel for accurate interpretation of the microscopy. These tests can take up to 2 days to complete.

Because current methods for detecting important infectious agents are not adequately sensitive or specific, there is a need for a method which would allow more sensitive and specific detection of two or more disease-causing or linked pathogens. A more sensitive and specific test for pathogens will permit appropriate therapy to be initiated earlier, when it may be more effective at reducing the length or severity of disease.

### SUMMARY

The present disclosure meets the foregoing need and allows detection of organisms using PCR, which results in a significant increase in accuracy and decrease in time, as well as other advantages apparent from the discussion herein. The present disclosure, in a general and overall sense, provides a unique method for detecting multiple pathogens and/or other contaminants in a sample containing a biological specimen. In some aspects, the method provides for the sensitive and specific detection of Giardia and Cryptosporidium. This method is therefore important in many applications, including clinical diagnosis of animal (human and non-human) pathologies and environmental (water and soil) monitoring. The method may also be used for monitoring or detection of target organisms in any other context, including, without limitation, veterinary tests; medical tests; air quality; biological warfare defense (including terrorism and weapons of mass destruction); hazardous waste cleanup; bioremediation; environmental reclamation, restoration, and cleanup; and the like.

According to some embodiments, a biological sample may be any specimen or sample capable of containing a pathogenic organism, such as Giardia, Cryptosporidium, Salmonella, Shigella, Campylobacter, Candida, E. coli, Yersinia, Aeromonas, Microsporidia or other small pathogenic organism. A biological sample may include a sample obtained from a water supply; sewer treatment area; a soil sample from a farming area; animal grazing area; waste disposal area; and/or a sample obtained from virtually any water source used by animals or humans for consumption, cleaning, or any other domestic or commercial use; or the like. In addition, a biological sample may comprise human or animal waste materials (e.g., stool), medical refuse (bandages and wound dressings), body fluid (urine, plasma, blood, mucus, etc), and/or the like. In some embodiments, the methods provide for the screening and/or testing of a biological specimen such as drinking wafer and/or bodies of water (such as a stream, river, or lake) from which drinking water is obtained.

In some aspects, there is provided a pathogen detection method that requires 50% or less time to complete than conventional methods for measuring the same or similar pathogen. In some embodiments, the methods are also significantly more cost-effective than currently available methods. By way of example, the method is about 35% less expensive than currently available detection methods used for similar purposes, such as microscopic examination methods.

In another aspect, there is provided a method that is capable of genetically detecting two or more microorganisms in a sample. By way of example, such two or more microorganisms may comprise Giardia and Cryptosporidium. Among other advantages, this method is capable of detecting 1,000 pathogens or fewer per specimen, while conventional methods have reported sensitivities of 10,000-50,000 pathogens per specimen.

The present disclosure also provides for a detection protocol that may require less than two hours to complete. In some specific embodiments, the invention provides for water quality testing. This type of testing typically requires a relatively high volume filtration. Because the present methods rely on real-time PCR detection, which detects microorganism-specific (e.g., Cryptosporidium- and/or Giardia-specific) DNA sequences, a relatively high volume filtration may not be needed. In contrast to other water quality testing methods, the present methods do not rely on visual determination or antibody binding.

Commercial uses of the present methods include clinical diagnosis of a human stool specimen, veterinary diagnosis from an animal stool specimen, water quality testing from recreational or drinking water samples, and environmental testing from soil or other sample types.

The present disclosure provides a real time PCR assay which enables the individual detection of Giardia and Cryptosporidium. The present disclosure has the advantage over the prior art in that it can detect any combination of two (2) or more infectious agents, such as, e.g., Giardia and Cryptosporidium, without the use of antibodies (e.g., in traditional ELISA and IFA methodologies).

Accordingly, in one aspect of the disclosure, a nucleic acid-based method may be used to determine to presence of two or more microscopic pathogens in a sample. The method includes isolating nucleic acids, including DNA, from the sample to provide an isolate, placing a portion of the isolate in a reaction vessel, placing a PCR reaction mixture in the reaction vessel, placing primer nucleic acid sequences and probe nucleic acid sequences in the reaction vessel, placing an internal control nucleic acid sequence and probe internal control nucleic acid sequences in the reaction vessel, amplifying nucleic acid sequences in the isolate and in the internal control nucleic acid sequence using the primer nucleic acid sequences, and detecting probe nucleic acid sequences bound to amplified target nucleic acid sequences in the reaction vessel. The primer sequences are configured to amplify a target sequence in a species, and the probe sequences are configured to bind to the target sequence. The internal control is configured to bind at least two primers from the primer sequences. The probe internal control sequences are configured to bind to a unique target sequence in the internal control sequence. The target nucleic acid sequence in the species is preferentially amplified over the unique internal control target sequence. The presence of probe nucleic acid sequences bound to target nucleic acid sequences indicates the presence of the species. if the species is not present in the sample, then the internal control is amplified, and the presence of probe internal control sequences bound to the unique target nucleic acid sequence indicates an absence of PCR inhibition.

The internal control may include SEQ ID NO:9, and the probe internal control sequences may include SEQ ID NO:10 and SEQ ID NO:11. The method may further include placing a portion of the isolate in a second reaction vessel, placing a PCR reaction mixture in the second reaction vessel, placing second primer nucleic acid sequences and second probe nucleic acid sequences in the second reaction vessel, placing an internal control nucleic acid sequence and probe internal control nucleic acid sequences in the second reaction vessel, amplifying nucleic acid sequences in the isolate and in the internal control nucleic acid sequence using the primer nucleic acid sequences, and detecting probe nucleic acid sequences bound to amplified target nucleic acid sequences in the second reaction vessel. The second primer sequences are configured to amplify a target sequence in a second species, and the second probe sequences are configured to bind to the target sequence in the second species. The internal control is configured to bind at least two primers selected from the first primer sequences and the second primer sequences. The second target nucleic acid sequence in the second species is preferentially amplified over the unique internal control target sequence. The presence of second probe nucleic acid sequences bound to target nucleic acid sequences indicates the presence of the second species. If the second species is not present in the sample, then the internal control is amplified, and the presence of probe internal control sequences bound to the unique target nucleic acid sequence indicates an absence of PCR inhibition. The first species may be Giardia, and the second species may be Cryptosporidium. The primer nucleic acid sequences may include SEQ ID NO: 1, SEQ ID NO:2, SEQ ID NO:5, and SEQ ID NO:6. The probe nucleic acid sequences for Giardia may be SEQ ID NO:7 and SEQ ID NO:8, and the probe nucleic acid sequences for Cryptosporidium may be SEQ ID NO:3 and SEQ ID NO:4. The sample may be a stool sample or a water sample.

According to another aspect of the disclosure, a kit may be used to screen a sample for two or more biological contaminants. The kit includes a first primer pair, a first probe pair, a second primer pair, a second probe pair, an internal control, and an internal control probe pair. The first primer pair is configured to amplify a first target sequence in a first species, and the first probe pair is configured to detect the first target sequence. The second primer pair is configured to amplify a second target sequence in a second species, and the second probe pair is configured to detect a second target sequence. The internal control includes a first end region, an IC body, and a second end region. The first end region includes a sequence that is complementary to a forward primer of the first primer pair and a sequence that is complementary to a forward primer of the second primer pair. The second end region includes a sequence that is complementary to a reverse primer of the first primer pair and a sequence that is complementary to a reverse primer of the second primer pair. The internal control probe pair is configured to detect the internal control.

The internal control may include SEQ ID NO:9, and the probe internal control sequences may include SEQ ID NO:10 and SEQ ID NO:11. The first species may be Cryptosporidium, and the second species may be Giardia. The first primer pair sequences may include SEQ ID NO:1 and SEQ ID NO:2. The second primer pair sequences may include SEQ ID NO:5 and SEQ ID NO:6. The first probe pair sequences may include SEQ ID NO:3 and SEQ ID NO:4. The second probe pair sequences may include SEQ ID NO:7 and SEQ ID NO:8.

The components of the kit may be lyophilized and provided in one or more master mixes. The first master mix may include the first primer pair, the first probe pair, the internal control, and the internal control probe pair. The second master mix comprising the second primer pair, the second probe pair, the internal control, and the internal control probe pair. More specifically, the first master mix may include Tfi Buffer in the range of about 1× to about 5×, MgCl2 in the range of about 3 mM to about 10 mM, Trehalose in the range of about 0.1 M to about 0.5M, dATP in the range of about 0.1 mM to about 0.5 mM, dTTP in the range of about 0.1 mM to about 0.5 mM, dCTP in the range of about 0.1 mM to about 0.5 mM, dGTP in the range of about 0.1 mM to about 0.5 mM, Cryptosporidium Forward Primer in the range of about 0.2 μM to about 0.7 μM, Cryptosporidium Reverse Primer in the range of about 0.2 μM to about 0.7 μM, Cryptosporidium Donor Probe in the range of about 0.02 μM to about 0.4 μM, Cryptosporidium Acceptor Probe in the range of about 0.1 μM to about 0.3 μM, IC Donor Probe in the range of about 0.1 μM to about 0.5 μM, IC Acceptor Probe in the range of about 0.1 μM to about 0.5 μM, TFi DNA Polymerase in the range of about 0.05 U/μl to about 0.3 U/μl: and IC in the range of about 0.3 fg/μl to about 0.7 fg/μl. The second master mix may include Tfi Buffer in the range of about 1× to about 5×, MgCl2 in the range of about 3 mM to about 10 mM, Trehalose in the range of about 0.1 M to about 0.5M, dATP in the range of about 0.1 mM to about 0.5 mM, dTTP in the range of about 0.1 mM to about 0.5 mM, dCTP in the range of about 0.1 mM to about 0.5 mM, dGTP in the range of about 0.1 mM to about 0.5 mM, Giardia Forward Primer in the range of about 0.2 μM to about 1.0 μM, Giardia Reverse Primer in the range of about 0.2 μM to about 1.0 μM, Giardia Donor Probe in the range of about 0.1 μM to about 0.5 μM, Giardia Acceptor Probe in the range of about 0.1 μM to about 0.5 μM, IC Donor Probe in the range of about 0.1 μM to about 0.5 μM, IC Acceptor Probe in the range of about 0.1 μM to about 0.5 μM, TFi DNA Polymerase in the range of about 0.05 U/μl to about 0.3 U/μl: and IC DNA in the range of about 0.3 fg/μl to about 0.7 fg/μl.

In yet another aspect of the disclosure, composition includes a primer pair, a probe pair, an internal control, and an internal control probe pair. The primer pair is configured to amplify a target sequence in a species, and the probe pair is configured to detect the target sequence. The internal control includes a first end region, an IC body, and a second end region. The first end region includes a sequence that is complementary to a forward primer of the primer pair, and the second end region includes a sequence that is complementary to a reverse primer of the primer pair. The internal control probe pair is configured to detect the internal control. The primer pair may be either SEQ ID NO: 1 and SEQ ID NO:2 or SEQ ID NO:5 and SEQ ID NO:6. The internal control may include SEQ ID NO:9.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### DETAILED DESCRIPTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. This, for example, a reference to “a capsule” is a reference to one or more capsules and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, temperature, pressure, time and the like, is, for example, from 1 to 90, specifically from 20 to 80, more specifically from 30 to 70, it is intended that values such as 15 to 85, 22 to 68, 43 to 51, 30 to 32 etc., are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Moreover, provided below is a “Definitions” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

### DEFINITIONS

The term “amplification” of nucleic acids, including DNA, as used herein means the use of PCR to increase the concentration of a particular nucleic acid sequence within a mixture of nucleic acid sequences. The particular nucleic acid sequence that is amplified is described herein as a “target” sequence.

The term “any bond” as used herein, especially in reference to nucleic acid sequences, means any bond or configuration of atoms that will block 3′ chain extension of an oligonucleotide without interfering with the binding properties of the oligonucleotide.

The term “any link” as used herein, especially in reference to nucleic acid sequences, means that any appropriate linkage may be used. For example, a number of different are available to join a fluorophore to an oligonucleotide, including without limitations thiol linkages and amine linkages.

The terms “biological sample” and “sample” as used herein mean any specimen or sample of matter capable of containing an organism. Non-limiting examples include a sample of water, a soil sample, an air sample, a stool sample, a blood sample, a urine sample, and the like.

The term “Cryptosporidium” as used herein means any species of Cryptosporidium which is known to cause disease in humans including C. parvum, C. Jells, C. muris, C. meleagridis, C. suis, C. canis, and/or C. hominis.

The term “Giardia” as used herein by itself, not followed by a species name, means any species of Giardia which is known to cause disease in humans. This may include G. lamblia. G. duodenalis, and/or G. intestinalis.

The term “fluorophore” as used herein means a functional group attached to a nucleic acid that will absorb energy of a specific wavelength and re-emit energy at a different, but equally specific, wavelength.

The term “internal control” sequence as used herein refers to a nucleic acid sequence that may be used to demonstrate that a PCR reaction is functioning to detect a nucleic acid sequence.

The terms “pathogen,” “organism.” and “species” are used interchangeably herein and refer to any one species, or closely-related group of species, that may be uniquely identified by an oligonucleotide sequence. The species may be known or unknown and may include viruses.

The term “PCR” as used herein means the polymerase chain reaction, as is well-known in the art. The term includes all forms of PCR, such as, e.g., real-time PCR and quantitative PCR.

The term “positive control target DNA” means a nucleic acid containing a sequence known to be complementary to a probe or probe pair. Positive control target DNA may be used as a positive control to determine that the probe is correctly binding to its target.

The term “primer pair” as used herein means a pair of oligonucleotide primers that are complementary to the sequences flanking a target sequence. The primer pair consists of a forward primer and a reverse primer. The forward primer has a nucleic acid sequence that is complementary to a sequence upstream, i.e. 5′, of the target sequence. The reverse primer has a nucleic acid sequence that is complementary to a sequence downstream, i.e. 3′, of the target sequence.

The terms “probe” and “probe pair” refer to one or two oligonucleotide sequences that are complementary to a specific target sequence and are covalently linked to a fluorophore. A probe pair includes two oligonucleotides: a “donor probe” and an “acceptor probe.” When both probes are bound to the target sequence, the donor probe's fluorophore may transfer energy to the acceptor probe's fluorophore in a Förster resonance energy transfer (FRET).

The term “reaction vessel” as used herein means a container used for performing PCR and for detecting specific nucleic acid sequences.

The term “species under investigation” as used herein means one or more species suspected to be present in a sample, and the methods, procedures, and materials of the present disclosure are employed to determine whether or not the species is actually present.

The term “target” sequence as used herein means the sequence of a nucleic acid that is amplified by PCR.

### DESCRIPTION

According to an aspect of the present disclosure, the presence of one or more species may be detected in a sample. In particular, the disclosure is well suited to detecting two pathogens, but more or different types of organisms may be targeted without departing from the spirit and scope of the invention. For example, the disclosure permits testing for the presence or absence of Cryptosporidium and Giardia in a single sample.

Once a sample is collected, DNA may be isolated and extracted from the sample. The isolated DNA may be divided into small portions and placed in a reaction vessel, such as, e.g., a PCR tube, with appropriate PCR reagents. Each reaction vessel may also receive a pair of primers, a pair of oligonucleotide probes, an internal control (IC) construct, and a pair of probes for the internal control. The primers and probes may be specific for a single species under examination. The PCR reagents, primers, probes, and IC may be provided in a mixture or ready-to-use form, e.g., in a solution or as a freeze-dried mixture. The internal control may also be amplified by the species-specific primer, but it is detected with its own unique probes. With the availability of primer and probe pairs for multiple species, the isolate from a single sample may be tested for the presence of multiple species of interest.

In one aspect of the disclosure, a master mix may be prepared for each organism under investigation. For example, a master mix targeting Cryptosporidium may contain the following primers:

Cryptosporidium Forward Primer:
(SEQ ID NO: 1)
5′-AAT AAA TCA TAA GCC TAC CGT GGC AAT GA-3′
Cryptosporidium Reverse Primer:
(SEQ ID NO: 2)
5′-AAT AAA TCA TAA AAA GTC CTG TAT TGT TAT TTC
TTG TC-3′

An exemplary Cryptosporidium master mix may also contain the following probes:

Cryptosporidium Donor Probe:
(SEQ ID NO: 3)
5′-CGG CTA CCA CAT CTA AGG AAG GC-any link-any
low emitting fluorophore in range (green)-3′
Cryptosporidium Acceptor Probe:
(SEQ ID NO: 4)
5′-any high emitting fluorophore in range (red)-
CAG GCG CGC AAA TTA CCC AAT CCT A-any bond-3′

As an additional example, a master mix targeting Giardia may contain the following primers:

Giardia Forward Primer:
(SEQ ID NO: 5)
5′-AAT AAA TCA TAA GGA CGG CTC AGG ACA AC-3′
Giardia Reverse Primer:
(SEQ ID NO: 6)
5′-AAT AAA TCA TAA GGA GTC GAA CCC TGA TTC T-3′

A master mix targeting Giardia may also contain the following probes:

Giardia Donor Probe:
(SEQ ID NO: 7)
5′-CCT TGC GCG CAC GTC TTG-any link-any low
emitting fluorophore in range (green)-3′
Giardia Acceptor Probe:
(SEQ ID NO: 8)
5′-any high emitting fluorophore in range (red)-
CCG GTT GCC AGC GGT GT-any bond-3′

In an additional aspect of the disclosure, an internal control (IC) construct may be provided as part of a PCR master mix or as a separate component. The IC allows monitoring of PCR efficiency and inhibition. PCR inhibition is a particular concern with DNA isolated from stool samples, which may contain inhibitory compounds such as mucoglycoproteins and proteases. The internal control may be a double-stranded DNA construct. Starting at the 5′ end of the “sense” strand, the IC may include an end region 1, an IC body, and an end region 2. These regions may be immediately adjacent to one or other, or there may be spacer sequences between regions. End region 1, end region, 2 or both may be omitted as appropriate for a particular application. In one aspect of the disclosure, end region 1 may contain a sequence that is complementary to a forward primer for a species under investigation. End region 2 may contain a sequence that is complementary to a reverse primer for the same species. In an alternate aspect, each end region may contain multiple forward or reverse primers. For example, if two species are under investigation, each end region may contain one primer binding site for each species. It is possible to investigate more species, and thus include more primer binding sites, without departing from the spirit and scope of the disclosure.

For example, if the species under investigation are Giardia and Cryptosporidium, then end region may contain a binding site for a forward primer for Giardia and a binding site for a forward primer for Cryptosporidium. Similarly, end region 2 may contain a binding site for a reverse primer for Giardia and a binding site for a reverse primer for Cryptosporidium. By relying on species-specific primers to amplify the internal control, the IC may not require its own set of primers for amplifaction. A single construct and a single set of probes may be included in the master mix for each targeted species, thereby reducing costs and complexity. More important, reducing the number of oligonucleotides in each reaction vessel may improve PCR efficiency and reduces the chance for artifacts, preferential amplification, and other errors. Methods and assays according to the present disclosure may include a total of six oligonucleotides in each reaction vessel, for example, two Giardia primers, two Giardia probes, and two IC probes. As an additional example, the six oligonucleotides may include two Cryptosporidium primers, two Cryptosporidium probes, and two IC probes. Oligonucleotides may be joined to fluorophores using amine linkages, thiol linkages, or the like. In addition, oligonucleotides may have functional groups or bonds to block 3′ chain extension, such as phosphate bonds, C-3 spacer bonds, and the like.

According to additional aspects of the disclosure, the IC may be present at relatively low levels so that it does not out-compete any template that may be present from a species under investigation. In this situation, a species target sequence, if present, may be preferentially amplified instead of the IC. In other words, only the species may be amplified and detected, and the IC may not be amplified or detected. If the species target sequence is not present, however, then IC template may be amplified by the species primers, and the internal control may be detected by its own probes. In the case where neither internal control nor species target sequence is detected, there may be a problem with the PCR reaction, most likely inhibition of PCR by components of the sample.

By relying on an artificial sequence as internal control, the present disclosure eliminates problems inherent in other PCR assays for pathogen screening and detection. In particular, these assays typical amplify a human (or other species) gene present in a sample for their internal control. The gene may be present at high copy-numbers, which may mask a failure of PCR amplification, or the signal from the selected control gene may overwhelm any signal from the species under investigation. By using a small amount of artificial internal control DNA and preferentially amplifying either the species target sequence or the IC, the present disclosure reduces or removes these types of errors.

In some aspects of the disclosure, the IC body may have a length of 150 to 450 base pairs (bp). In some of these aspects, the IC body may have a length of 274 bp. In one particular aspect, the IC body may have the following sequence:

(SEQ ID NO: 9)
5′-GCC TAC CGT GGC AAT GAA GGA CGG CTC AGG ACA
ACT TCT GAC TTT TGT CGT GCT GTG CGA CAC GTA AAT
TTA GTC CCC CAA TAA ATA ACA GGC CGC TGT TGA GCA
CAA GCA GCT AGC GCC GTT TTA GCC ACA TGT ACC CAG
TAT ATA TGT CAC GAG AGG ATA GGC GAA CGG ATG CTG
ACG AGG CAA CAA AAG CAC AGG TAC TCG AGG GAA GGT
TGG AAT GGT CAG GCC GAC AAG AAA TAA CAA TAC AGG
ACT TTA AGA ATC AGG GTT CGA CTC C-3′

According to an aspect of the disclosure, the probes far the IC construct may have the following sequences:

Internal Control donor probe:
(SEQ ID NO: 10)
5′-CGG ATG CTG ACG AGG CAA CA-any low emitting
fluoophore in range (green)-any link-3′
Internal Control acceptor probe:
(SEQ ID NO: 11)
5′-any high emitting fluorophore in range (red)-
GCA CAG GTA CTC GAG GGA AGG-any bond-3′

In some aspects of the disclosure, the fluorophores of the various acceptor probes may be selected so that the IC probe emits at a different wavelength than a species-specific probe. The following illustrations are for exemplary purposes only, and any variation known to one skilled in the art may be practiced without departing from the spirit and scope of the disclosure and the claims. For example, the species acceptor probe may be fitted with a mid-range red fluorophore, such as, e.g., Alex fluor 680, which emits at 680 nm, while the IC acceptor probe may be linked to a high-emission red fluorophore, such as, e.g., LC 705, which emits at a wavelength of 705 nm.

In general, a donor probe may be linked to a fluorophore at its 3′ end, thereby preventing the probe from acting like a primer during PCR. In addition, an acceptor probe usually sits 3′ of the donor, further blocking chain extension. An acceptor probe, however, may be free at its 3′ end. According to some aspects of the disclosure, an acceptor probe may be blocked at its 3′ end to prevent it from acting as a primer during PCR. Functional groups or bonds to block 3′ chain extension include phosphate bonds, C-3 spacer bonds, and the like.

As will be understood by one of ordinary skill in the art, the present disclosure may utilize a Förster resonance energy transfer (FRET) for the detection of target DNA. The FRET transfer may take place between a low-emitting fluorophore attached to the 3′ end of a donor probe and a second, high-emitting fluorophore attached to the 5′ end of the corresponding acceptor probe. For example, low-emitting fluorophores may emit light with a wavelength of 400-500 nm, and high-emitting fluorophores may emit light with a wavelength of 580-710 nm. Other arrangements of fluorophores and donor and acceptor probes are contemplated and are within the scope and spirit of the disclosure. By way of example only, the role of the donor probe and acceptor probe may be reversed. In this example, the sequences of the oligonucleotides stay the same, but the acceptor has a fluorophore at its 3′ end and binds upstream, i.e. 5′, of the donor and the donor has a fluorophore at its 5′ end. In some aspects, the donor probe may include a green fluorophore, and the acceptor probe may include a red fluorophore. Examples of suitable green fluorophores may include, without limitation, FAM, FITC, Alexa fluor 488, or the like. Examples of suitable red fluorophores may include, without limitation, LC 705, Texas Red, Alexa fluor 680, or the like. An emission by a red fluorophore may be detected in channel 3 of the LightCycler 2.0, as well as on other common PCR platforms, such as ABI 7300/7500, Corbett Roto gene, Finnzyme qPCR platform, BioRad iCycler, and the like.

According to additional aspects of the disclosure, the IC may be used to monitor the efficiency of DNA extraction techniques. Poor DNA extraction can occur due to incomplete cell lysis, DNA degradation, or inefficient binding to the purification matrix. For example, the double-stranded DNA of the IC construct may be inserted into a generic plasmid and transformed into E. coli for cloning. The transformed E. coli clones may then be used to spike a stool specimen prior to DNA extraction. Once DNA has been extracted and isolated from the sample, the isolate may be tested to determine the presence and amount of IC in the isolate. Testing may be performed, e.g., using quantitative PCR.

While the invention has been described in terms of exemplary aspects, those skilled in the art will recognize that the invention can be practiced with modifications in the spirit and scope of the appended claims. These examples given above are merely illustrative and are not meant to be an exhaustive list of all possible designs, aspects, applications, or modifications of the invention.

### SPECIFIC EXAMPLES

The following specific examples are indicative of preferred aspects of the present disclosure, but they are provided for illustrative purposes only. One of ordinary skill in the art will understand that the following illustrative examples may be modified for a particular application within the spirit and scope of the claims.

### Example 1

### Product for Pathogen Detection

The present example is directed to a description of the product as it exists in the format of different modules, the specific modules depending on the end use of the test and/or the PCR platform being used. For example, in some embodiments, 2 modules may be included. These modules may include: (1) DNA extraction reagents and consumables; and/or (2) PCR detection reagents and protocol.

The DNA extraction reagents will vary depending upon the starting material to provide optimized extractions for each type of starting material. The PCR detection reagents and protocol will also vary depending upon the starting material and/or the PCR platform used for the assay, providing optimized reagents and protocol for at least, for example, 4 major PCR platforms.

The final product may incorporate: (1) Sensitive DNA extraction methodology with reagents customized specifically for the end use, and/or (2) Sensitive, optimized PCR reagents with internal control and positive control target DNA, usable on, e.g., Roche LightCycler, Cepheid SmartCycler, ABI 7300/7500, Corbett Roto gene, Finnzyme qPCR platform, BioRad iCycler, or the like.

### Example 2

### Cryptosporidium/Giardia real-time PCR Protocol (LightCycler-Roche)

The present example is provided to demonstrate a protocol that may be used in the analysis of a specimen suspected to be infected or to contain two (2) or more environmental pathogens, such as Cryptosporidium and Giardia. The following presents the step-by-step method by which the diagnostic test of a sample of interest may be run.

### 1. Setup LightCycler

a. Turn on thermocycler.

b. Turn on the computer and open the LightCycler software.

c. Follow the standard software menu options to load or create an experiment file.

### 2. Setup PCR Reactions

a. All reagents should be kept cold and protected from light at all times.
b. Obtain the correct number of Cryptosporidium-specific master mixes, prepared during manufacturing with the following components and then freeze-dried:

| Component | Concentration |
|---|---|
| Tfi Buffer | 1x-5x |
| MgCl₂ | 3 mM-10 mM |
| Trehalose | 0.1 M-0.5 M |
| dATP | 0.1 mM-0.5 mM |
| dTTP | 0.1 mM-0.5 mM |
| dCTP | 0.1 mM-0.5 mM |
| dGTP | 0.1 mM-0.5 mM |
| Cryptosporidium Forward Primer | 0.2 μM-0.7 μM |
| Cryptosporidium Reverse Primer | 0.2 μM-0.7 μM |
| Cryptosporidium Donor Probe | 0.02 μM-0.4 μM |
| Cryptosporidium Acceptor Probe | 0.1 μM-0.3 μM |
| IC Donor Probe | 0.1 μM-0.5 μM |
| IC Acceptor Probe | 0.1 μM-0.5 μM |
| TFi DNA Polymerase | 0.05 U/μl-0.3 U/μl |
| IC | 0.3 fg/μl-0.7 fg/μl |

The Cryptosporidium forward primer may have the sequence specified by SEQ ID NO: 1. The Cryptosporidium reverse primer may halve the sequence specified by SEQ ID NO: 2. The Cryptosporidium donor probe may have the sequence specified by SEQ ID NO: 3. The Cryptosporidium acceptor probe may have the sequence specified by SEQ ID NO: 4. The IC DNA may have the sequence specified by SEQ ID NO: 9. The IC donor probe may have the sequence specified by SEQ ID NO: 10, and the IC acceptor probe may have the sequence specified by SEQ ID NO: 11.
c. Obtain the correct number of Giardia-specific master mixes, prepared during manufacturing with the following components and then freeze-dried:

| Component | Concentration |
|---|---|
| Tfi Buffer | 1x-5x |
| MgCl₂ | 3 mM-10 mM |
| Trehalose | 0.1 M-0.5 M |
| dATP | 0.1 mM-0.5 mM |
| dTTP | 0.1 mM-0.5 mM |
| dCTP | 0.1 mM-0.5 mM |
| dGTP | 0.1 mM-0.5 mM |
| Giardia Forward Primer | 0.2 μM-1.0 μM |
| Giardia Reverse Primer | 0.2 μM-1.0 μM |
| Giardia Donor Probe | 0.1 μM-0.5 μM |
| Giardia Acceptor Probe | 0.1 μM-0.5 μM |
| IC Donor Probe | 0.1 μM-0.5 μM |
| IC Acceptor Probe | 0.1 μM-0.5 μM |
| TFi DNA Polymerase | 0.05 U/μl-0.3 U/μl |
| IC DNA | 0.3 fg/μl-0.7 fg/μl |

The Giardia forward primer may have the sequence specified by SEQ ID NO: 5. The Giardia reverse primer may have the sequence specified by SEQ ID NO: 6. The Giardia donor probe may have the sequence specified by SEQ ID NO: 7, and the Giardia acceptor probe may have the sequence specified by SEQ ID NO: 8. The IC DNA may have the sequence specified by SEQ ID NO: 9. The IC donor probe may have the sequence specified by SEQ ID NO: 10, and the IC acceptor probe may have the sequence specified by SEQ ID NO: 11.
d. Obtain a tube of reconstitution buffer, consisting of the following components:

| Component | Concentration |
|---|---|
| Molecular Biology-Grade H₂O | 90-100% |
| Dimethyl Sulfoxide, 99% pure | 0-10% |

e. Reconstitute the Cryptosporidium- and Giardia-specific master mixes with the volume of reconstitution buffer specified in the product insert. Mix well and centrifuge briefly.
f. Pipette 17 μl of the appropriate master mix into each glass capillary.
g. Add 3 μl of molecular biology-grade H₂O to each negative control capillary and cap.
h. Add 3 μl of the positive control target DNA to each positive control capillary and cap.
i. Add 3 μl of the appropriate sample DNA to each open capillary and cap.
j. Load capillaries into a LightCycler carousel and centrifuge capillaries using the procedure recommended in the product insert.
k. Load the carousel into the LightCycler and close the lid.

### 3. Run PCR Reactions

a. Input the following run parameters into the LightCycler control software:

| | | | |
|---|---|---|---|
| | | | Acquisition |
| Step | Temp (° C.) | Time (min:sec) | Mode |

Denaturation (Cycles: 1, Analysis: None)

| | | | |
|---|---|---|---|
| Hot Start | 90-95 | 01:00-15:00 | None |

Amplification (Cycles: 35-50, Analysis: Quantification)

| | | | |
|---|---|---|---|
| Denature | 90-95 | 00:05-00:25 | None |
| Anneal | 50-60 | 00:10-00:30 | Single |
| Extend | 70-75 | 00:20-00:40 | None |

Melting Curve (Cycles: 1, Analysis: Melting Curves)

| | | | |
|---|---|---|---|
| Denature | 90-95 | 00:00-00:10 | None |
| Anneal | 40-50 | 00:20-00:40 | None |
| Melting | 80-85(slope = 0.1° C./sec) | 00:00-00:10 | Continuous |

Cooling (Cycles: 1, Analysis: None)

| | | | |
|---|---|---|---|
| Cooling | 40 | 00:20-00:40 | None |

b. Enter the appropriate sample information into the LightCycler control software.

c. Save the run conditions.

d. Click “Run” to begin the PCR amplification run

### 4. Analyze Data

a. The data analysis module will open automatically upon completion of the run.
b. Select the appropriate color compensation file as specified in the product insert.
c. The appropriate quantification and melt curve data may now be viewed and printed using the LightCycler Control Software.

What is claimed is:

1. A nucleic acid-based method for determining the presence of at least two microscopic pathogens in a sample, the method comprising:
isolating nucleic acids from the sample to provide an isolate, the nucleic acids comprising DNA:
placing a portion of the isolate in a first reaction vessel;
placing a PCR reaction mixture in the first reaction vessel;
placing first primer nucleic acid sequences and first probe nucleic acid sequences in the first reaction vessel, the first primer sequences configured to amplify a target sequence in a first species, the first probe sequences configured to bind to the target sequence in the first species;
placing an internal control nucleic acid sequence and probe internal control nucleic acid sequences in the first reaction vessel, the internal control configured to bind at least two primers selected from the first primer sequences, the probe internal control sequences configured to bind to a unique target sequence in the internal control sequence;
amplifying nucleic acid sequences in the isolate and in the internal control nucleic acid sequence using the primer nucleic acid sequences; and
detecting probe nucleic acid sequences bound to amplified target nucleic acid sequences in the first reaction vessel, wherein the presence of first probe nucleic acid sequences bound to target nucleic acid sequences indicates the presence of the first species, the presence of probe internal control sequences bound to the unique target nucleic acid sequence indicates an absence of PCR inhibition.

2. The method of claim 1, wherein the internal control comprises a sequence
(SEQ ID NO: 9)
5′-GCC TAC CGT GGC AAT GAA GGA CGG CTC AGG ACA
ACT TCT GAC TTT TGT CGT GCT GTG CGA CAC GTA AAT
TTA GTC CCC CAA TAA ATA ACA GGC CGC TGT TGA GCA
CAA GCA GCT AGC GCC GTT TTA GCC ACA TGT ACC CAG
TAT ATA TGT CAC GAG AGG ATA GGC GAA CGG ATG CTG
ACG AGG CAA CAA AAG CAC AGG TAC TCG AGG GAA GGT
TGG AAT GGT CAG GCC GAC AAG AAA TAA CAA TAC AGG
ACT TTA AGA ATC AGG GTT CGA CTC C-3′.

3. The method of claim 1, wherein the probe internal control sequences are
IC Donor Probe:
(SEQ ID NO: 10)
5′-CGG ATG CTG ACG AGG CAA CA-any link-any low
emitting fluorophore in range (green)-3′;
and
IC Acceptor Probe:
(SEQ ID NO: 11)
5′-Any high emitting fluorophore in range (red)-
GCA CAG GTA CTC GAG GGA AGG-any bond-3′.

4. The method of claim 1, further comprising:
placing a portion of the isolate in a second reaction vessel;
placing a PCR reaction mixture in the second reaction vessel;
placing second primer nucleic acid sequences and second probe nucleic acid sequences in the second reaction vessel, the second primer sequences configured to amplify a target sequence in a second species, the second probe sequences configured to bind to the target sequence in the second species;
placing an internal control nucleic acid sequence and probe internal control nucleic acid sequences in the second reaction vessel, the internal control configured to bind at least two primers selected from the first primer sequences and the second primer sequences, the probe internal control sequences configured to bind to a unique target sequence in the internal control sequence;
amplifying nucleic acid sequences in the isolate and in the internal control nucleic acid sequence using the primer nucleic acid sequences; and
detecting probe nucleic acid sequences bound to amplified target nucleic acid sequences in the second reaction vessel, wherein the presence of second probe nucleic acid sequences bound to target nucleic acid sequences indicates the presence of the second species, the presence of probe internal control sequences bound to the unique target nucleic acid sequence indicates an absence of PCR inhibition.

5. The method of claim 4, wherein the first species is Giardia and the second species is Cryptosporidium.

6. The method of claim 5, wherein the primer nucleic acid sequences are
Giardia forward primer:
(SEQ ID NO: 5)
5′-AAT AAA TCA TAA GGA CGG CTC AGG ACA AC-3′;
Giardia reverse primer:
(SEQ ID NO: 6)
5′-AAT AAA TCA TAA GGA GTC GAA CCC TGA TTC T-3′;
Cryptosporidium forward primer:
(SEQ ID NO: 1)
5′-AAT AAA TCA TAA GCC TAC CGT GGC AAT GA-3′;
and
Cryptosporidium reverse primer:
(SEQ ID NO: 2)
5′-AAT AAA TCA TAA AAA GTC CTG TAT TGT TAT TTC
TTG TC-3′.

7. The method of claim 5, wherein the probe nucleic acid sequences for Giardia are
Giardia donor probe:
(SEQ ID. NO. 7)
5′-CCT TGC GCG CAC GTC TTG-any link-any low
emitting fluorophore in range (green)-3′;
Giardia acceptor probe:
(SEQ ID NO: 8)
5′-any high emitting fluorophore in range (red)-
CCG GTT GCC AGC GGT GT-any bond-3′;
and
further wherein the probe nucleic acid sequences for Cryptosporidium are
Cryptosporidium donor probe:
(SEQ ID NO: 3)
5′-CGG CTA CCA CAT CTA AGG AAG GC-any link-any
low emitting fluorophore in range (green)-3′;
Cryptosporidium acceptor probe:
(SEQ ID NO: 4)
5′-any high emitting fluorophore in range (red)-
CAG GCG CGC AAA TTA CCC AAT CCT A-any bond-3′.

8. The method of claim 1, wherein the sample is a water sample or a stool sample.

9. A kit for screening a sample for at least two biological contaminants, the kit comprising:
a first primer pair configured to amplify a first target sequence in a first species:
a first probe pair configured to detect the first target sequence;
a second primer pair configured to amplify a second target sequence in a second species;
a second probe pair configured to detect a second target sequence:
an internal control comprising a first end region, an IC body, and a second end region, the first end region comprising a sequence that is complementary to a forward primer of the first primer pair and a sequence that is complementary to a forward primer of the second primer pair, the second end region comprising a sequence that is complementary to a reverse primer of the first primer pair and a sequence that is complementary to a reverse primer of the second primer pair; and
an internal control probe pair configured to detect the internal control.

10. The kit of claim 9, wherein the internal control comprises a sequence
(SEQ ID NO: 6)
5′-GCC TAC CGT GGC AAT GAA GGA CGG CTC AGG ACA
ACT TCT GAC TTT TGT CGT GCT GTG CGA CAC GTA AAT
TTA GTC CCC CAA TAA ATA ACA GGC CGC TGT TGA GCA
CAA GCA GCT AGC GCC GTT TTA GCC ACA TGT ACC CAG
TAT ATA TGT CAC GAG AGG ATA GGC GAA CGG ATG CTG
ACG AGG CAA CAA AAG CAC AGG TAC TCG AGG GAA GGT
TGG AAT GGT CAG GCC GAC AAG AAA TAA CAA TAC AGG
ACT TTA AGA ATC AGG GTT CGA CTC C-3′.

11. The kit of claim 9, wherein the internal control probe pair sequences are
IC Donor Probe:
(SEQ ID NO: 10)
5′-CGG ATG CTG ACG AGG CAA CA-any link-any low
emitting fluorophore in range (green)-3′;
and
IC Acceptor Probe:
(SEQ ID NO: 11)
5′-Any high emitting fluorophore in range (red)-
GCA CAG GTA CTC GAG GGA AGG-any bond-3′.

12. The kit of claim 9, wherein the first species is Cryptosporidium and the second species is Giardia.

13. The kit of claim 12, wherein the first primer pair sequences are
Cryptosporidium forward primer:
(SEQ ID NO: 1)
5′-AAT AAA TCA TAA GCC TAC CGT GGC AAT GA-3′;
Cryptosporidium reverse primer:
(SEQ ID NO: 2)
5′-AAT AAA TCA TAA AAA GTC CTG TAT TGT TAT TTC
TTG TC-3′;
and
further wherein the second primer pair sequences are
Giardia forward primer:
(SEQ ID NO: 5)
5′-AAT AAA TCA TAA GGA CGG CTC AGG ACA AC-3′;
and
Giardia reverse primer:
(SEQ ID NO: 6)
5′-AAT AAA TCA TAA GGA GTC GAA CCC TGA TTC
T-3′.

14. The kit of claim 12, wherein the first probe pair sequences are
Cryptosporidium donor probe:
(SEQ ID NO: 3)
5′-CGG CTA CCA CAT CTA AGG AAG GC-any link-any
low emitting fluorophore in range (green)-3′;
Cryptosporidium acceptor probe:
(SEQ ID NO: 4)
5′-any high emitting fluorophore in range (red)-
CAG GCG CGC AAA TTA CCC AAT CCT A-any bond-3′;
and
further wherein the second probe pair sequences are
Giardia donor probe:
(SEQ ID. NO. 7)
5′-CCT TGC GCG CAC GTC TTG-any link-any low
emitting fluorophore in range (green)-3′;
and
Giardia acceptor probe:
(SEQ ID NO: 8)
5′-any high emitting fluorophore in range (red)-
CCG GTT GCC AGC GGT GT-any bond-3′.

15. The kit of claim 9, wherein the components are lyophilized and provided in a first master mix and a second master mix,
the first master mix comprising the first primer pair, the first probe pair, the internal control, and the internal control probe pair; and
the second master mix comprising the second primer pair, the second probe pair, the internal control, and the internal control probe pair.

16. The kit of claim 15, wherein the first master mix comprises:
Tfi Buffer in the range of about 1× to about 5×;
MgCl₂ in the range of about 3 mM to about 10 mM:
Trehalose in the range of about 0.1 M to about 0.5M;
dATP in the range of about 0.1 mM to about 0.5 mM;
dTTP in the range of about 0.1 mM to about 0.5 mM,
dCTP in the range of about 0.1 mM to about 0.5 mM:
dGTP in the range of about 0.1 mM to about 0.5 mM;
Cryptosporidium Forward Primer in the range of about 0.2 μM to about 0.7 μM;
Cryptosporidium Reverse Primer in the range of about 0.2 μM to about 0.7 μM;
Cryptosporidium Donor Probe in the range of about 0.02 μM to about 0.4 μM;
Cryptosporidium Acceptor Probe in the range of about 0.1 μM to about 0.3 μM;
IC Donor Probe in the range of about 0.1 μM to about 0.5 μM;
IC Acceptor Probe in the range of about 0.1 μM to about 0.5 μM;
TFi DNA Polymerase in the range of about 0.05 U/μl to about 0.3 U/μl; and
IC in the range of about 0.3 fg/μl to about 0.7 fg/μl.

17. The kit of claim 15, wherein the second master mix comprises:
Tfi Buffer in the range of about 1× to about 5×;
MgCl₂ in the range of about 3 mM to about 10 mM;
Trehalose in the range of about 0.1 M to about 0.5M;
dATP in the range of about 0.1 mM to about 0.5 mM;
dTTP in the range of about 0.1 mM to about 0.5 mM;
dCTP in the range of about 0.1 mM to about 0.5 mM;
dGTP in the range of about 0.1 mM to about 0.5 mM;
Giardia Forward Primer in the range of about 0.2 μM to about 1.0 μM;
Giardia Reverse Primer in the range of about 0.2 μM to about 1.0 μM;
Giardia Donor Probe in the range of about 0.1 μM to about 0.5 μM;
Giardia Acceptor Probe in the range of about 0.1 μM to about 0.5 μM;
IC Donor Probe in the range of about 0.1 μM to about 0.5 μM;
IC Acceptor Probe in the range of about 0.1 μM to about 0.5 μM;
TFi DNA Polymerase in the range of about 0.05 U/μl to about 0.3 U/μl; and
IC DNA in the range of about 0.3 fg/μl to about 0.7 fg/μl.

18. A composition comprising:
a primer pair configured to amplify a target sequence in a species;
a probe pair configured to detect the target sequence;
an internal control comprising a first end region, an IC body, and a second end region, the first end region comprising a sequence that is complementary to a forward primer of the primer pair, the second end region comprising a sequence that is complementary to a reverse primer of the primer pair; and
an internal control probe pair configured to detect the internal control.

19. The composition of claim 18, wherein the primer pair is selected from the following primer pairs:
Cryptosporidium forward primer:
(SEQ ID NO: 1)
5′-AAT AAA TCA TAA GCC TAC CGT GGC AAT GA-3′
and
Cryptosporidium reverse primer:
(SEQ ID NO: 2)
5′-AAT AAA TCA TAA AAA GTC CTG TAT TGT TAT TTC
TTG TC-3′;
and
Giardia forward primer:
(SEQ ID NO: 5)
5′-AAT AAA TCA TAA GGA CGG CTC AGG ACA AC-3′
and
Giardia reverse primer:
(SEQ ID NO: 6)
5′-AAT AAA TCA TAA GGA GTC GAA CCC TGA TTC
T-3′.

20. The composition of claim 18, wherein the internal control comprises a sequence
(SEQ ID NO: 6)
5′-GCC TAC CGT GGC AAT GAA GGA CGG CTC AGG ACA
ACT TCT GAC TTT TGT CGT GCT GTG CGA CAC GTA AAT
TTA GTC CCC CAA TAA ATA ACA GGC CGC TGT TGA GCA
CAA GCA GCT AGC GCC GTT TTA GCC ACA TGT ACC CAG
TAT ATA TGT CAC GAG AGG ATA GGC GAA CGG ATG CTG
ACG AGG CAA CAA AAG CAC AGG TAC TCG AGG GAA GGT
TGG AAT GGT CAG GCC GAC AAG AAA TAA CAA TAC AGG
ACT TTA AGA ATC AGG GTT CGA CTC C-3′.


SEQUENCE LISTING (11 sequences)

SEQ ID NO: 1 - DNA, 29 bp, Cryptosporidium parvum
aataaatcataagcctaccgtggcaatga

SEQ ID NO: 2 - DNA, 38 bp, Cryptosporidium parvum
aataaatcataaaaagtcctgtattgttatttcttgtc

SEQ ID NO: 3 - DNA, 23 bp, Cryptosporidium parvum
cggctaccacatctaaggaaggc

SEQ ID NO: 4 - DNA, 25 bp, Cryptosporidium parvum
caggcgcgcaaattacccaatccta

SEQ ID NO: 5 - DNA, 29 bp, Giardia intestinalis
aataaatcataaggacggctcaggacaac

SEQ ID NO: 6 - DNA, 31 bp, Giardia intestinalis
aataaatcataaggagtcgaaccctgattct

SEQ ID NO: 7 - DNA, 18 bp, Giardia intestinalis
ccttgcgcgcacgtcttg

SEQ ID NO: 8 - DNA, 17 bp, Giardia intestinalis
ccggttgccagcggtgt

SEQ ID NO: 9 - DNA, 274 bp, Artificial Sequence
  Internal Control artificial sequence
gcctaccgtggcaatgaaggacggctcaggacaacttctgacttttgtcgtgctgtgcgacacgtaaatttagtcccccaataaataacaggccgctgttgagcacaagcagctagcgccgttttagccacatgtacccagtatatatgtcacgagaggataggcgaacggatgctgacgaggcaacaaaagcacaggtactcgagggaaggttggaatggtcaggccgacaagaaataacaatacaggactttaagaatcagggttcgactcc

SEQ ID NO: 10 - DNA, 20 bp, Artificial Sequence
  Donor probe for internal control construct
cggatgctgacgaggcaaca

SEQ ID NO: 11 - DNA, 21 bp, Artificial Sequence
  Acceptor probe for internal control construct
gcacaggtactcgagggaagg